(19) United States
(12) Patent Application Publication
(10) Pub. No.: US 2026/0151605 A1
(43) Pub. Date: Jun. 4, 2026

(54) Hydrogen Sulfide Delivery Device

(71) Applicant: Exhalix LLC, Albuquerque, NM (US)

(72) Inventors: Alireza Shekarriz, Cle Elum, WA (US); Debra May Friedrichsen, Corvallis, OR (US); Bill Jay Brooks, Lebanon, OR (US)

(21) Appl. No.: 19/123,464

(22) PCT Filed: Oct. 26, 2023
(86) PCT No.: PCT/US2023/077974

§ 371 Date: Apr. 23, 2025

Related U.S. Application Data
(60) Provisional application No. 63/420,454, filed on Oct. 28, 2022.

(51) Int. Cl. A61M 35/00; A61B 17/00; A61B 17/06; A61F 13/00; A61K 33/04; A61K 41/00; A61L 15/44; A61M 1/00; C25B 1/01

(52) U.S. Cl. CPC A61M 35/30; A61B 17/06166; A61F 13/00063; A61K 33/04; A61K 41/00; A61L 15/44; A61M 1/94; C25B 1/01; A61B 2017/00893; A61F 2013/00357

(57) ABSTRACT

A hydrogen sulfide generator includes an electrolytic cell. The cell includes first and counter-electrodes, an electrolyte and a solid-state hydrogen sulfide precursor in electrical connection with the first electrode. Upon applying a small potential across the cell, the hydrogen sulfide precursor generates hydrogen sulfide gas which permeates from the cell. The hydrogen sulfide generator is useful for delivering therapeutic levels of hydrogen sulfide to wounded tissue, and this is a useful component of a tissue dressing.

This work was supported by the United States National Institutes of Health under grant no. 1R43GM144027-01. The United States Government has certain rights to this invention.

There are circumstances in which it would be useful to provide small but controlled quantities of hydrogen sulfide. For example, hydrogen sulfide sensors are sometimes used in industrial and waste treatment operations to detect leaks or otherwise monitor exposure to hydrogen sulfide gas. These sensors often require calibration, and for that small and controlled amounts of hydrogen sulfide gas are needed. These quantities of hydrogen sulfide gas can be provided in the form of bottled gas and/or as liquid solutions, but this requires packaging and transporting the gas and its containers, as well as delivery apparatus that can include pumps, valves and other complex apparatus. There is also the danger of leakage from the packaging, and the difficulty in metering out very small quantities as are often needed for calibration purposes.

Hydrogen sulfide has also been identified as a vascularization promoter for animal, particular mammalian (including human) tissue. For this purpose, very small amounts need to be delivered in a controllable manner. It is further desirable to deliver the hydrogen sulfide directly to the tissue, at the site where vascularization and/or healing promotion is wanted. The tissue is often not directly accessible for hydrogen sulfide treatment, being covered by a dressing, or perhaps being subcutaneous tissue.

In each case, a way by which hydrogen sulfide can be generated in very small quantities on demand using simple, easily controllable apparatus is wanted. In one aspect, the invention is a hydrogen sulfide generator comprising an electrolytic cell, the electrolytic cell comprising A) a first electrode, B) at least one solid-state sulfur-containing hydrogen sulfide precursor in electrical contact with the first electrode, (C) a counter-electrode spaced apart from (i) the first electrode and (ii) the at least one solid-state sulfur-containing hydrogen sulfide precursor in electrical contact with the first electrode, and D) an electrolyte reservoir disposed between the first and counter-electrode, wherein (i) the hydrogen sulfide generator is permeable to hydrogen sulfide produced by the electrolytic cell such that hydrogen sulfide produced by the electrolytic cell is permeable out of at least one surface of the hydrogen sulfide generator, (ii) the at least one solid-state sulfur-containing hydrogen sulfide precursor is insoluble in the electrolyte and chemically stable in the presence of the electrolyte in the absence of an applied voltage to the electrolytic cell and (iii) the hydrogen sulfide generator is permeable to liquid electrolyte solution such that liquid electrolyte solution contacting at least one surface of the hydrogen sulfide generator permeates into the electrolytic cell to become disposed between the first and counter-electrode.

The invention in a second aspect is a hydrogen sulfide generator comprising an electrolytic cell, the electrolytic cell comprising A) a first electrode, B) at least one solid-state sulfur-containing hydrogen sulfide precursor in electrical contact with the first electrode, (C) a counter-electrode spaced apart from (i) the first electrode and (ii) the at least one solid-state sulfur-containing hydrogen sulfide precursor in electrical contact with the first electrode, and D) an electrolyte disposed between the first and counter-electrode, wherein the hydrogen sulfide generator is permeable to hydrogen sulfide produced by the electrolytic cell such that hydrogen sulfide produced by the electrolytic cell is permeable out of at least one surface of the hydrogen sulfide generator, wherein the at least one solid-state sulfur-containing hydrogen sulfide precursor is insoluble in the electrolyte and chemically stable in the presence of the electrolyte in the absence of an applied voltage to the electrolytic cell.

In a third aspect, the invention is a hydrogen sulfide generator, comprising:
I) a gel electrolyte devoid of sulfur-containing hydrogen sulfide precursor;
II) first and counter-electrodes at least partially embedded in the gel electrolyte and separated by the gel electrolyte;
III) at least one solid-state sulfur-containing hydrogen sulfide precursor in electrical contact with the first electrode and separated from the counter-electrode by the gel electrolyte;
IV) optionally at least one solid-state sulfur-containing hydrogen sulfide precursor in electrical contact with the counter-electrode and separated from the first electrode by the gel electrolyte,
   wherein the gel electrolyte forms at least a portion of the exterior surface of the hydrogen sulfide generator and/or at least a portion of the exterior surface of the gel electrolyte is covered with a hydrogen sulfide-permeable surface layer that forms an exterior surface of the hydrogen sulfide generator.

In a fourth aspect, the invention is a tissue dressing having a tissue-contact side and an opposing side, the tissue dressing comprising a hydrogen sulfide generator of any of the preceding aspects, wherein at least a portion of a surface of the tissue-contact side of the tissue dressing is permeable to hydrogen sulfide produced by the electrolytic cell of the hydrogen sulfide generator.

In a fifth aspect, the invention is a tissue treatment method, comprising:
a) applying a tissue dressing of the fourth aspect to living tissue with the tissue-contact side of the tissue dressing in contact with the living tissue;
b) applying a voltage across the electrolytic cell of the tissue dressing to convert at least a portion of the sulfur-containing hydrogen sulfide precursor to hydrogen sulfide; and
c) actively or passively permeating the hydrogen sulfide through the tissue-contact side of the tissue dressing and into contact with the living tissue.

In a sixth aspect, the invention is an implantable tissue dressing comprising a hydrogen sulfide generator of the third aspect.

In a seventh aspect, the invention is a tissue treatment method, comprising:
a) implanting an implantable tissue dressing of the sixth aspect into living tissue; and
b) applying a voltage across the electrolytic cell of the implantable tissue dressing to convert at least a portion of the sulfur-containing hydrogen sulfide precursor to hydrogen sulfide.

In an eighth aspect, the invention is a hydrogen sulfide generator, comprising:
A) a first wire electrode having at least one solid-state sulfur-containing hydrogen sulfide precursor incorporated into and/or forming a layer on a surface of the first wire electrode, wherein at least a portion of the first wire electrode or solid-state sulfur-containing hydrogen sulfide precursor is exposed at a surface of the hydrogen sulfide generator;
C) a wire counter-electrode; and
D) a gel electrolyte separating the first wire electrode and the wire counter-electrode.

In a ninth aspect, the invention is a suture comprising a hydrogen sulfide generator of the eighth aspect.

### BRIEF DESCRIPTION OF THE DRAWINGS

FIG. 1 is a perspective sectional view of an embodiment of a hydrogen sulfide generator of the invention.

FIG. 2 is a perspective sectional view of a second embodiment of a hydrogen sulfide generator of the invention.

FIG. 3 is a perspective sectional view of a third embodiment of a hydrogen sulfide generator of the invention.

FIG. 4 is an isometric view, partially in section, of an electrode for use in the invention.

FIG. 5 is an isometric view, partially in section, of another embodiment o of an electrode for use in the invention.

FIG. 6 is a cross-sectional view of a composite electrically conductive particle for use in an electrode for use in the invention.

FIG. 7 is a perspective view of an electrode/solid-state hydrogen sulfide precursor generator for use in the invention.

FIG. 7A is a cross-sectional view of an electrically conductive fiber for use in the invention.

FIG. 8 is a front view, partially in section, of an electrode coated encapsulated a gel layer that contains hydrogen sulfide precursor.

FIG. 8A is a perspective view of an electrode coated coated with a gel layer that contains hydrogen sulfide precursor.

FIG. 9 is a perspective view, partially in section, of a tissue dressing of the invention.

FIG. 10 is a sectional view of a tissue dressing of the invention implanted in living tissue.

FIG. 11 is a front view of another embodiment of a hydrogen sulfide generator of the invention.

FIG. 12 is a sectional view of an embodiment of a hydrogen sulfide generator of the invention useful as an implantable tissue dressing.

The thicknesses of the various layers shown in the Figures are exaggerated for purposes of illustration.

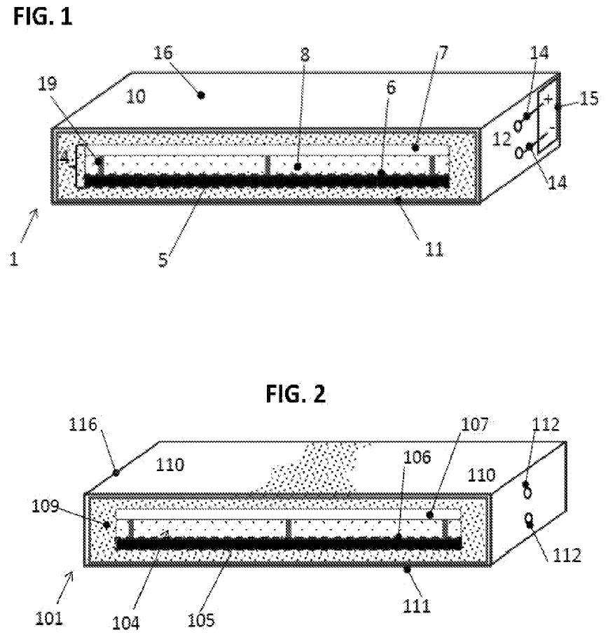

Turning to FIG. 1, hydrogen sulfide generator 1 of the invention includes electrolytic cell 4. Electrolytic cell 4 includes first electrode 5 and counter-electrode 7. Solid-state hydrogen sulfide precursor 6 is in electrical contact with first electrode 5. Electrolyte 8 is disposed between first electrode 5 and counter-electrode 7 and in contact with each. In the embodiment shown in FIG. 1, optional spacer 19 is provided. When optional spacer 19 is present, it may be, for example, an open-celled foam, mesh, woven or nonwoven fabric, hydrogel, swellable polymer or other material that holds electrolyte 8.

In the embodiment shown in FIG. 1, electrolytic cell 4 is contained with an optional housing 16. As shown, housing 16 includes outer seal 10 and bottom element 11, which together enclose electrolytic cell 4. Housing 16, when present, may perform any or all or several functions. It may, for example, form a barrier to a liquid electrolyte, to prevent leakage; perform a protective function, provide electrical insulation, provide a scaffold or support structure for electrolytic cell 4 and/or one or more components thereof; and/or provide a scaffold or support for various other components of hydrogen sulfide generator 1 or other device (such as a tissue dressing) that includes the hydrogen sulfide generator 1 as a component, and provide the means to attach and/or seal to a sensor for calibration or to a tissue dressing for delivery of hydrogen sulfide. In the embodiment shown in FIG. 1, bottom element 11 is a physical barrier to the electrolyte and other liquids.

Hydrogen sulfide generator 1 is permeable to hydrogen sulfide produced by electrolytic cell 4 such that during operation hydrogen sulfide produced by electrolytic cell 4 permeates out of at least one surface thereof.

In reference to hydrogen sulfide, a structure (such as hydrogen sulfide generator 1 in FIGS. 1 and 1 A, electrolytic cell 4 and/or housing 16, or any component thereof such as outer seal 10 and bottom element 11) is “permeable” to hydrogen sulfide if the structure allows hydrogen sulfide to pass through it by bulk flow (such as bulk movement of a gas, liquid, or another medium containing hydrogen sulfide), molecular diffusion, or a combination of both mechanisms. In some embodiments, the hydrogen sulfide generator is permeable to hydrogen sulfide only at predefined areas that constitute only a portion of its surface (such as a single side or portion of a single side). This allows, for example, hydrogen sulfide permeating from the hydrogen sulfide generator to escape in a defined and predetermined direction. For example, when the hydrogen sulfide generator is present in a tissue dressing, a tissue-contact side of the tissue dressing may be permeable to hydrogen sulfide, whereas other surfaces are not, to direct hydrogen sulfide onto bodily tissues to which the tissue dressing is applied. In embodiments such as shown in FIG. 1, bottom element 11 is selectively permeable, being permeable to hydrogen sulfide but impermeable to liquids including, for example, the electrolyte (if a liquid) and water, whereas outer seal 10 is impermeable to both hydrogen sulfide and liquids. In embodiments such as shown in FIG. 1, hydrogen sulfide produced during operation permeates out of hydrogen sulfide generator 1 only through bottom element 11.

The electrolyte may or may not be sealed within the hydrogen sulfide generator of the invention. In the embodiment shown in FIG. 1, electrolyte 8 is sealed within hydrogen sulfide generator 1.

A second embodiment of the hydrogen sulfide generator is illustrated in FIG. 2. Hydrogen sulfide generator 101 of the invention includes electrolytic cell 104. Electrolytic cell 104 includes first electrode 105 and counter-electrode 107. Electrolyte reservoir 109 is disposed between first electrode 105 and counter-electrode 107. Solid-state hydrogen sulfide precursor 106 is in electrical contact with first electrode 105. During operation, electrolyte reservoir 109 holds electrolyte. In this embodiment, electrolytic cell 104 is contained within housing 116 which, as shown, includes optional but preferred outer seal 110 and bottom element 111. Housing 116 performs functions similar to described with regard to housing 16 in FIG. 1, with the exception that at least a portion of housing 116 in this embodiment is permeable to liquid electrolyte solution. In the embodiment shown in FIG. 2, bottom element 111 is permeable to hydrogen sulfide, and also permeable to a liquid electrolyte solution such that liquid electrolyte solution contacting bottom element 111 permeates into electrolytic cell 104 to become disposed in electrolyte reservoir 109 between first electrode 105 and counter-electrode 107. Solid-state sulfur-containing hydrogen sulfide precursor 106 is in electrical contact with first electrode 105. Electrolyte reservoir 109 may contain a spacer, which may comprise, for example, a swellable polymer or dehydrated hydrogel material which then swells and/or rehydrates upon being contacted with liquid electrolyte solution.

In the embodiment shown in FIG. 2, electrolyte is not sealed within hydrogen sulfide generator 101. In embodiments such as shown in FIG. 2, in which electrolyte is not sealed within the hydrogen sulfide generator, electrolyte can flow into and out of the electrolytic cell, which offers significant manufacturing, storage/shipment and operational advantages. Electrolyte can be supplied at the time of use, rather than being incorporated into the electrolytic cell at the time of manufacture. This simplifies manufacturing, reduces weight for shipment, and eliminates risks of leakage during shipment and storage. Electrolyte in such cases can be supplied at the time of usage. For example, a liquid electrolyte solution can be applied to a liquid-permeable surface of the hydrogen sulfide generator, such as bottom element 111 of FIG. 2. The liquid electrolyte solution then permeates through the liquid-permeable surface and into the reservoir of the electrolytic cell.

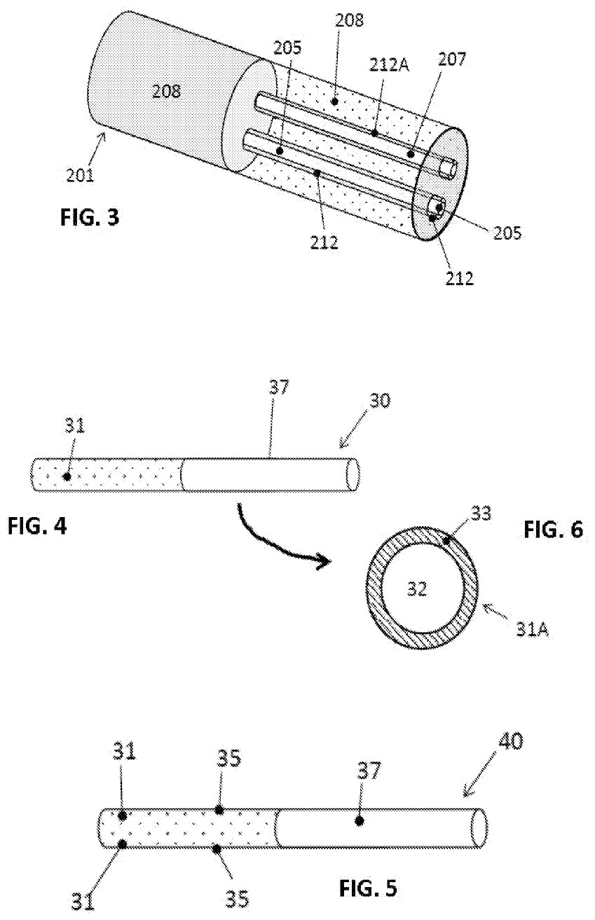

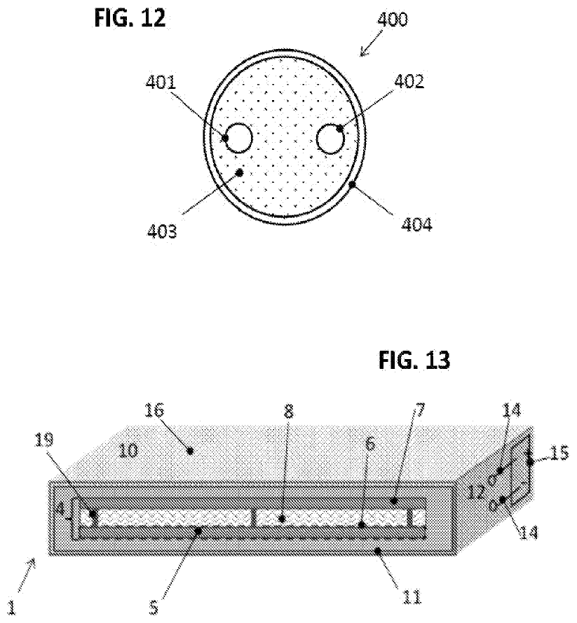

A third embodiment of the hydrogen sulfide generator of the invention is shown in FIG. 3. Hydrogen sulfide generator 201 comprises gel electrolyte 208, which is devoid of sulfur-containing hydrogen sulfide precursor and permeable to hydrogen sulfide. Gel electrolyte may be biocompatible, bioresorbable or both biocompatible and bioresorbable. First electrode 205 and counter-electrode 207 are embedded in and separated by gel electrolyte 208. Solid-state sulfur-containing hydrogen sulfide precursor 212 is in electrical contact with first electrode 205; in the embodiment shown, hydrogen sulfide precursor 212 is applied to the surface of first electrode 205. The particular embodiment shown in FIG. 3, optional separator 212A is interposed between first electrode 205 and counter electrode 207. As shown, separator 212A is applied as a coating or sheath on counter electrode 207, but when present may be positioned anywhere between first electrode 205 and counter electrode 207. Additional solid-state sulfur-containing hydrogen sulfide precursor may be in electrical contact with counter electrode 207. Gel electrolyte 208 separates solid-state sulfur-containing hydrogen sulfide precursor 212 and electrode 205 from counter-electrode 207 (and any hydrogen sulfide precursor in electrical contact with counter electrode 207) In the embodiment shown, gel electrolyte 208 forms the exterior surface of hydrogen sulfide generator 201. Alternatively, some or all of the exterior surface of gel electrolyte 208 may be covered with a hydrogen sulfide-permeable surface layer (such as tube 404 in FIG. 12) that forms an exterior surface of hydrogen sulfide generator 201. For example, a portion but less than all of the exterior surface of gel electrolyte is covered with a hydrogen sulfide-impermeable surface layer (not shown) that forms part but not all of the exterior surface of hydrogen sulfide generator 201. Alternatively, all of the exterior surface of gel electrolyte may be covered with a selective, hydrogen sulfide-permeable surface layer (not shown) that forms all of the exterior surface of hydrogen sulfide generator 201 and fluidically contains the electrolytic cell, but is only permeable to hydrogen sulfide.

Yet in another embodiment, a dielectric spacer may be present to keep the spacing between the electrodes 205 and 212 fixed along the length of the electrodes. Such a spacer may be or include a separator as described below.

The first and counter-electrodes comprise electrically conductive materials, such as metals, conductive carbons, intrinsically conductive polymers such as poly(pyrroles), poly(thiphene)s, poly(3,4-ethylendioxythiphene), poly(p-phenylene sulfide) poly(acetylene)s, poly(p-phenylene-vinylene)s, polyanilines, and the like.

In some embodiments, one or both of the first and counter-electrode(s) comprises electrically conductive particles embedded in a binder, such as is shown in FIGS. 4 and 5. Suitable binders include, for example, organic polymers (which may be conductive, semi-conductive or non-conductive, thermoplastic or thermoset), ceramic materials, pastes and densely-packed suspensions, silicone materials, and the like. Other suitable binders include hydrogels, i.e., three-dimensional polymeric networks containing entrapped water. The hydrogel may be amorphous, semi-crystalline, hydrogen-bonded and/or supramolecular, and may be chemically (such as covalently) crosslinked and/or virtually crosslinked by physical entanglements, electrostatic attractive forces, hydrogen bonding, or the like. Examples of hydrogel materials include poly(vinyl alcohol) crosslinked with aldehydes to form acetal or hemiacetals; agar-based hydrogels; poly(ethylene oxide hydrogels; potassium poly(acrylate) hydrogels; gelatin hydrogels; inorganic hydrogels; polyacrylamide hydrogels; peptide-based hydrogels; and the like. If the binder in such an embodiment is not conductive, the electrically conductive particles should be present in a concentration and distribution that provides particle-to-particle contact. In some embodiments, the binder is biocompatible, bioresorbable, or both biocompatible and bioresorbable. Biocompatible and/or bioresorbable polymers include, for example, poly(glycolides), poly(lactides), poly(glycolide-co-lactide), poly-ε-caprolactone, other aliphatic polyesters, polyanhydrides, poly(ortho esters) (POE), polyphosphazenes, poly(amino acids) and ‘pseudo’ poly(amino acids), polyalkylcyanoacrylates, poly(propylene fumarate) (PPF), poloxamers, poly(p-dioxanone) (PPDO) and polyvinyl alcohol (PVA).

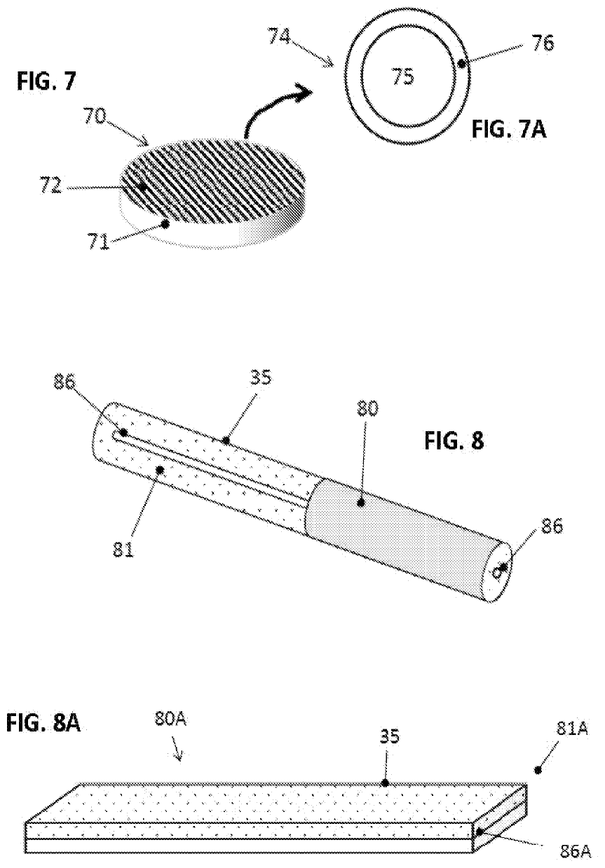

The physical geometry of the electrodes is not particularly critical and may be adapted to accommodate the specific design and end-use requirements of the hydrogen sulfide generator. Thus, the electrodes may be provided in the form of, for example, wires (as in FIGS. 4 and 5), plates, sheets or foils (as in FIG. 8A), meshes, grids, arrays, or any other convenient form. In a particular embodiment, the first electrode and optionally the counter-electrode is in the form of metallic wires or filaments embedded within a woven or non-woven fabric, the woven or non-woven fabric being permeable to hydrogen sulfide by bulk transfer and preferably permeable to liquids such as a liquid electrolyte, as shown, for example, in FIG. 7.

The first and counter-electrode may be of the same materials of construction and of the same geometry or other design features, or may be made of different materials and/or have different geometries and/or other design features.

A solid-state sulfur-containing hydrogen sulfide precursor is in electrical contact with the first electrode. For purposes of this invention, being in “electrical contact” means that during operation, electrons flow from the first electrode (5, 105, 205, in FIGS. 1-3) to the hydrogen sulfide precursor (6, 106, 212 in FIGS. 1-3). The solid-state sulfur-containing hydrogen sulfide precursor may be, for example, in direct physical contact with the first electrode, and/or may be separated only by one or more material materials that are electrically conductive, i.e., allow electrons to pass therethrough.

In some embodiments, the first electrode and hydrogen sulfide precursor are in direct physical contact, as shown, for example, in FIGS. 1, 2, 3 and 7. In the embodiments shown in FIGS. 1-3, hydrogen sulfide precursor 6, 106, 212 is present in the form of a layer, deposit, coating, or cladding in direct contact with a surface of first electrodes 5, 105, 205, respectively.

FIGS. 4 and 6 illustrate an alternative embodiment of an electrode having a heterogenous hydrogen sulfide precursor coating or layer for use in the invention. In FIG. 4, electrode 30 includes polymeric binder 37, which forms a matrix into which are embedded particles 31 and/or 31A (FIG. 6). Particles 31 and/or 31A preferably are packed close together within electrode 30 to provide particle-to-particle contact. Particles 31 are particles of an electrically conductive material as described before. Instead of or in addition to particles 31, polymeric binder contains composite particles such as composite particles 31A (FIG. 6), which comprise a core of electrically conductive material 32 and an exterior shell 33 of solid-state hydrogen sulfide precursor. Composite particles 31A can have other arrangements of the conductive material and solid-state hydrogen sulfide precursor. Primary particles of electrically conductive material and solid-state hydrogen sulfide precursor may be agglomerated to form composite particles. Instead of having the bilayer structure shown in FIG. 6, composite particles may contain a larger number of layers. Composite particles may include a binder phase to hold an electrically conductive material and hydrogen sulfide precursor together. Composite particles such as composite particles 31A may be porous to allow hydrogen sulfide generated during operation to escape.

FIG. 5 illustrates yet another alternative embodiment of an electrode for use in the invention. In FIG. 5, electrode 40 comprises particles 31 and/or 31A, as described in connection with FIGS. 4 and 6, and particles 35 of the solid-state hydrogen sulfide precursor, each preferably randomly distributed within polymeric binder 37, and preferably being packed close together within electrode 30 to provide particle-to-particle contact.

An embodiment of an electrode/solid-state hydrogen sulfide precursor assembly is illustrated in both FIGS. 8 and 8A. In FIG. 8, electrode 86, which is shown in the form of a wire, is encapsulated in and in direct contact with gel layer 81. Gel layer 81 includes gel 80, which forms a continuous or semi-continuous binder phase that contains and immobilizes particles 35 of solid-state hydrogen sulfide precursor. Gel layer 81 may alternatively cover only a portion of the surface of electrode 86, such as is illustrated by the embodiment shown in FIG. 8A. In FIG. 8A, electrode 86A is shown in the form of a sheet or film, and gel layer 81A is disposed only a single major surface of electrode 86A. As before, gel layer 81A includes gel 80A and particles 35 of solid-state hydrogen sulfide precursor. Another gel layer 81A of course may be applied to the opposing major surface of electrode 86A in this embodiment, and any gel layer 81A may be continuous or discontinuous and may cover only a portion of the surface of electrode 86A to which it is applied.

Gel layers 81 and 81A are preferably a hydrogel, including those described above a binder of the first and/or second electrode.

Instead of gel layer 81, hydrogen sulfide precursor may be provided in the form of a mesh wrapped around electrode 86, such as a fabric mesh impregnated with the hydrogen sulfide precursor or having the hydrogen sulfide precursor deposited thereon.

FIG. 7 illustrates an embodiment of an electrode/solid-state hydrogen sulfide precursor assembly that is particularly suitable when the hydrogen sulfide generator forms all or part of a tissue dressing. Electrode/solid-state hydrogen sulfide precursor assembly 70 comprises support 71 having electrical conductor 72 affixed to one major surface thereof. Such an electrode/solid-state hydrogen sulfide precursor assembly may function as the first electrode and an element of a housing for the electrolytic cell.

Support 71 is permeable to hydrogen sulfide and more preferably is also permeable to a liquid such as a liquid electrolyte solution. Support 71 is preferably not electrically conductive. Support 71 may include, for example, one or more of: a gauze or other fabric made up of natural and/or synthetic fibers; a mesh; a porous sheet or film; a polymeric foam, preferably hydrophilic, and the like. Such a gauze, fabric, mesh, porous sheet or film or foam may be impregnated with a hydrogen or a hydrogel precursor that forms a hydrogel upon contact with an aqueous fluid such as a liquid electrolyte solution (such as an exudate from living tissue). The material of construction may be selected from a wide range of materials, such as cotton, wool, rayon, a polyester, a polyolefin, a silicone, a nylon, silk, a polyurethane, a polylactide, as well as many others.

Electrical conductor 72 functions as an electrode of the hydrogen sulfide generator of the invention. Solid-state hydrogen sulfide precursor forms a surface layer on all or part of electrical conductor 72.

Electrical conductor 72 preferably is permeable to hydrogen sulfide and more preferably also permeable to liquids such as a liquid electrolyte solution. In the particular embodiment shown, electrical conductor 72 comprises electrically conductive woven, knotted (including knitted) or entangled metal or metallized fibers 74, which form a fabric and function as an electrode. In the embodiment shown, some or all of fibers 74 have core 75 of a metal or metallized fiber, and surface layer 76 of solid-state hydrogen sulfide precursor covering all or a part of the surface of core 75, which in this embodiment is in direct contact therewith. Electrical conductor 72 may, alternatively, take other physical forms such as a mesh, perforated metal, sheet or film, perforated metallized plastic sheet or film and the like. In any of those alternative form, a layer of solid-state hydrogen sulfide precursor is in direct contact with at least a portion of the surface of electrical conductor 72.

Electrical conductor 72 is shown as a discrete layer in FIG. 7. Alternatively, or in addition, the electrical conductor may be incorporated into support 71 such as by, for example, being provided in the form of electrically conductive wires, fibers, mesh, perforated sheet or plate, etc., incorporated into the body of support 71.

An electrode/solid-state hydrogen sulfide precursor assembly comprising a support, electrical conductor and surface layer of solid-state hydrogen sulfide precursor can be prepared in some cases by providing a starting support/electrical conductor assembly, in which the electrical assembly comprises a metal M. All or a part of the surface of the metal M is contacted with a composition comprising an alkali metal sulfide and/or alkali metal polysulfide (such as liver of sulfur) to convert all or a portion of such surface to the corresponding metal sulfide MₓS_{y}, x and y being integers that balance the charges, MₓS_{y} being the solid-state hydrogen sulfide precursor. A similar process can be used to form solid-state hydrogen sulfide precursor layers of the form MₓS_{y} on other metal electrode materials comprising the metal M.

Various commercially available silver metal-containing tissue dressing products are useful as starting support/electrical assemblies. These include, for example, those sold by Argentum Medical, LLC and bearing the Silverton® tradename, including the Silverton® Easy AG-22, Silverton® Island Wound and Surgical Dressing, and Silverton® Catheter Dressings, Hydrafoam Ag™ hydrophilic foam dressing sold by DermaRite Industries LLC and various products sold by Urgo Medical North America under the UrgoTul™ Ag tradename, including those sold in an absorptive dressing format, an alginate dressing format, a collagen dressing format, a composite dressing format, a foam dressing format, a gauze or non-woven dressing format, a gelling fiber dressing format, a hydrogen dressing format, an impregnated dressing format, a transparent film dressing format and a would filler dressing format. A solid-state silver sulfide layer can be formed onto the silver portion of product by treatment with liver of sulfur or other composition containing an alkali metal sulfide or polysulfide, in the general manner described before.

The solid-state hydrogen sulfide precursor is a compound or compounds that contain sulfur atoms, which decompose or otherwise react to produce hydrogen sulfide when a voltage is applied across the electrolytic cell. The solid-state hydrogen precursor may be, for example, a metal sulfide such as silver sulfide (Ag₂S), copper (II) sulfide (CuS), zinc sulfide (Zn₂S or ZnS), iron (II) sulfide (FeS), iron sulfur proteins including those described in Nature Communications 12, Article number 5925(2021 ), titanium (IV) sulfide (TiS₂), nickel (II) sulfide (NiS), selenium sulfide (SeS) and tin (II) sulfide (SnS). Among these, silver sulfide, copper sulfide, nickel sulfide and zinc sulfide are preferred, with silver sulfide being of particular interest, especially for tissue dressing applications in which infection control may also be of interest for efficient wound healing.

The electrolyte is a medium containing ions, is electrically conducting through the movement of those ions, but does not conduct electrons. With regard to the hydrogen sulfide generator of the first aspect, a wide range of electrolytes are useful, including liquid electrolytes, gel electrolytes, hydrogel-polymer electrolytes, polymer electrolytes, ceramic electrolytes, organic plastic electrolytes, polymer-in-ceramic electrolytes, ceramic-in-polymer electrolytes, room temperature ionic liquids or other types, or some combination of two or more of the foregoing types. Although any of these electrolyte types are useful in connection with the hydrogen sulfide of the second aspect of the invention, liquid or gel electrolytes are generally preferred in that case, especially liquid electrolytes, as liquid electrolytes and to a lesser extent gel electrolytes (or gelation of the electrolyte) can be introduced into the electrolyte reservoir at the time of use. A biocompatible gel electrolyte is present in the hydrogen sulfide generator of the third aspect.

Liquid electrolytes include, for example, aqueous solutions of protic mineral acids or organic acids such as sulfuric, phosphoric, nitric acid and acetic acid; and aqueous solutions of water-soluble salts, including aqueous solutions of one or more of alkali metal hydroxides, alkali metal halides, alkaline earth halides, alkali metal and alkaline earth carbonates, alkali metal nitrates, alkali metal sulfates, alkali metal phosphates, and the like. Saline solutions, which may be pH buffered, are useful liquid electrolytes.

Exudate from living tissues can also function as all or part of the electrolyte; accordingly, the electrolyte in tissue dressings of the invention may consist of or include living tissue exudate. In such a case, the tissue dressing may be applied to the tissue, after which tissue exudate seeps from the tissue into the electrolytic cell, supplying the electrolyte and/or replacing electrolyte that may be lost during use due to, for example, leakage and/or dehydration.

Gel electrolytes include hydrogels as described above with respect to electrode binders, which contain dissolved ions, such as dissolved mineral acids and/or water-soluble salts as described before.

In embodiments in which the electrolytic cell is contained within a housing (such as housing 16 in FIG. 1 and housing 116 in FIG. 2, the material(s) of construction of the housing may include, for example, organic polymers, which may be rigid, semi-flexible or flexible; metals; ceramics, biopolymers such as starch and cellulose; polysiloxanes; or other suitable material. An outer seal such as outer seal 10 of FIG. 1 and outer seal 110 of FIG. 2 preferably in some embodiments is impermeable to hydrogen sulfide and liquid (especially water and any liquid electrolyte solution as may be present), and is in addition preferably electrically insulating or else is not in electrical contact with the first and counter-electrodes. In some embodiments, all or part of the housing, such as bottom element 11 in FIG. 1 and bottom element 111 in FIG. 2, is hydrogen sulfide-permeable but liquid impermeable membrane, such as a polysiloxane or other hydrogen sulfide-permeable membrane. Such a membrane may exhibit a permeability (at 23° C.) to hydrogen sulfide of, for example, at least 1.75×10⁻⁴ picomol/s-m²-(Pa/m) (about 0.5 Barrer), at least 3.5×10⁻⁴ picomol/s-m²-(Pa/m) (about 1 Barrer), at least 0.175 picomol/s-m²-(Pa/m) (about 500 Barrer) or at least 0.35 picomol/s-m²-(Pa/m) (about 1000 Barrer). In embodiments in which, the membrane has a permeability to hydrogen sulfide at 23° C. of at most 1.75 picomol/s-m²-(Pa/m) (about 5000 Barrer), or to at most 1.05 picomol/s-m²-(Pa/m) (about 3000 Barrer) to at most 175×10⁻⁴ picomol/s-m²-(Pa/m) (about 50 Barrer), or to at most 70×10⁻⁴ picomol/s-m²-(Pa/m) (about 20 Barrer). Permeability can be measured using a volumetric method as described in ASTM D1434.

In alternative embodiments, all or part of the housing, such as bottom element 11 in FIG. 1 and bottom element 111 in FIG. 2, is permeable to both hydrogen sulfide and liquids, particularly water and an aqueous electrolyte solution. Materials as described with regard to support 71 in FIG. 7 are suitable. A separator, such as separator 212A in FIG. 3, may be present in any of the embodiments of the invention. Such a separator preferably is porous and allows ions to be transported therethrough. A separator may partially or completely envelop one or both of the first and counter electrodes, as shown in FIG. 3, or may have alternative geometries such as a sheet. The separator, when present, is interposed between the first and second electrodes. The separator should be a dielectric or non-conductive material. The material of construction may be selected from a wide range of materials, such as cotton, wool, rayon, a polyester, a polyolefin, a silicone, a nylon, silk, a polyurethane, a polylactide, as well as many others. The separator may take the form of a gauze or other fabric made up of natural and/or synthetic fibers; a mesh; a porous sheet or film; a polymeric foam, and the like.

The hydrogen sulfide generator produces hydrogen sulfide when a voltage is applied across the electrolytic cell. In particular, a negative charge is applied to the first electrode, which then operates as a cathode, supplying electrons to the solid-state hydrogen sulfide precursor, causing a chemical reaction that produces hydrogen sulfide. The potential across the electrolytic cell may be, for example, 0.1 to 2.0 volts, preferably 0.2 to 1.5 volts.

In embodiments in which hydrogen sulfide precursor is also in electrical connection with the counter-electrode, hydrogen sulfide can be produced at either electrode by operating either electrode as the cathode. For example, in one mode of operation, hydrogen sulfide is produced at either the first or the counter-electrode by applying a negative charge to the that electrode, causing it to operate as a cathode. Then, by reversing polarity, the other electrode is operated as the cathode and hydrogen sulfide is then produced at such other electrode. This manner of operation can increase the total amount of hydrogen sulfide produced by the hydrogen sulfide generator, because when the hydrogen sulfide precursor in electrical communication with one electrode is depleted, additional hydrogen sulfide can be generated at the other electrode.

Hydrogen sulfide produced by the hydrogen sulfide generator permeates out of the hydrogen sulfide generator.

Accordingly, the hydrogen sulfide generator of the invention preferably includes apparatus for supplying such a voltage (such as power source 15 in FIG. 1, which is in electrical contact with first electrode 5 and second electrode via wiring 14 and electrical contacts 12) and/or means for connecting the electrolytic cell to such an apparatus (such as a cord, wiring, electrical contacts such as contacts 112 of FIG. 2 and/or plug or plug receptacle).

Suitable apparatus for applying a voltage across the electrolytic cell include, for example, one or more of a potentiostat, a dipotentiostat, a polypotentiostat, a galvanostat, a buss regulator, AC/DC power converter, and a battery, in electrical connection with the first electrode and counter-electrode. A number of commercially available galvanometers and potentiostats are useful. An example of a suitable potentiostat is a Model 273-A potentiostat/galvanostat from Princeton Applied Research, Oak Ridge, Tennessee, operated with CorrWare software (from Scribner Associates, Southern Pines, North Carolina). Another suitable potentiometer is a Custom Sensor Solutions model 1401 potentiostat. Preferred devices are capable of imposing a voltage of from −2.5 to 2.5, especially from −0.5 to 1.5 volts, and of measuring currents in the range of from 1 nA to 100 mA, especially from 10 nA to 1 mA.

The electrolytic cell may also include electronic circuitry for measuring electrical conditions (typically current) produced during operation. A simple galvanometer or potentiostat is suitable for accomplishing both of these things. The circuitry may be, for example, an analog circuit that includes a pair of op amps, one functioning as a biased emitter follower to provide the desired bias voltage to the cell, and the other as a signal amplifier to measure the current produced by the cell. The circuitry may be digitally controlled, which facilitates real-time control of the applied voltage, baseline offsets and signal amplifier gain. Preferred devices are capable of measuring currents in the range of from 1 nA to 100 mA, especially from 10 nA to 1 mA.

Optional electronic circuitry may further include embedded circuitry or computing capability which permits a data processing algorithm to be incorporated directly into the instrument, without relying on an external computer. Alternatively, the hydrogen sulfide generator may include data transfer apparatus such that data and/or control instructions can be transferred to and/or from a separate device for processing the data, generating calibration curves, and the like.

The electrolytic cell of the invention preferably includes or is during use in electrical connection with at least one human-readable display. The display can be a visual type, a sonic type or some other suitable type. Combinations of various types can be used.

The hydrogen sulfide generator of the invention is useful for providing controlled amounts of hydrogen sulfide. It is useful for providing hydrogen sulfide gas for calibrating instruments such as hydrogen sulfide detectors.

Another application for the hydrogen sulfide generator is as a component of a living tissue dressing. Hydrogen sulfide has been related to improved vascularization and/or healing when applied to a wound site. The hydrogen sulfide generator of the invention can be operated to provide hydrogen sulfide in therapeutic quantities of, for example, 100 picomoles to 100 millimoles per day on a continuous basis and/or at different delivery intervals. A tissue dressing containing the hydrogen sulfide dressing, when applied to living tissue, delivers hydrogen sulfide directly to the tissue, where it can be absorbed and metabolized to provide vascularization and/or healing. The tissue dressing operates at such low voltages and amperages as are easily tolerated by the living tissue.

Accordingly, certain aspects of the invention are tissue dressings comprising the hydrogen sulfide generator. The tissue dressing may be, for example, a wound or surgical dressing, a catheter dressing, and/or an implantable dressing, and/or a surgical suture.

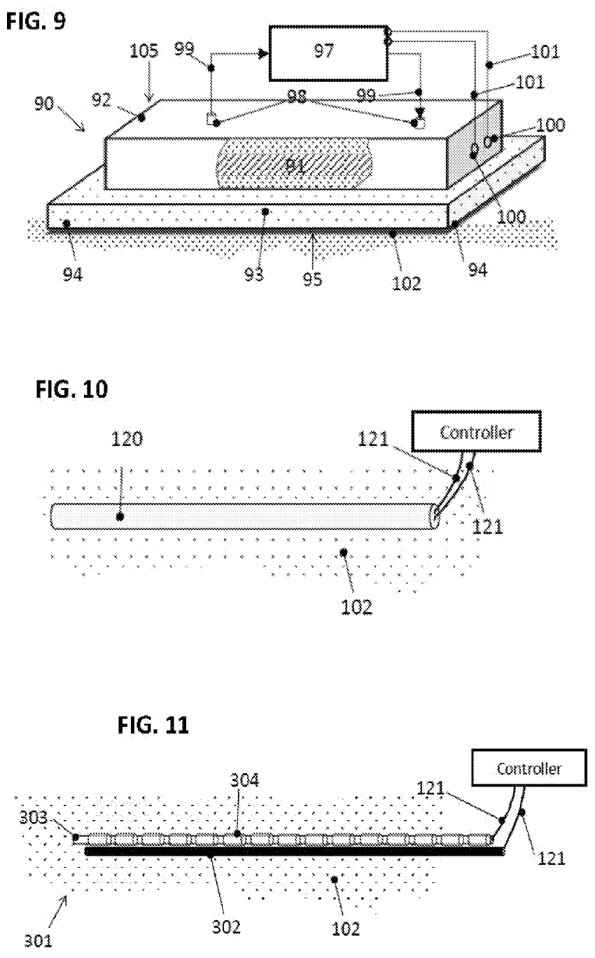

FIG. 9 illustrates an embodiment of a tissue dressing 90 of the invention, which is suitable for topical application, i.e., onto the skin or other exposed living tissue. Tissue dressing 90 has tissue-contact side 95 and opposing side 105. During use, tissue-contact side 95 is in contact with living tissue 102. Hydrogen sulfide generator 91 is disposed in tissue dressing 90. As shown, hydrogen sulfide generator 91 is disposed above bottom element 93, but as described before, bottom element 93 may be integrated into hydrogen sulfide generator 91 and form part thereof, such as a support for the first and/or counter-electrode, as described before.

Tissue-contact side 95 is permeable to hydrogen sulfide (as a gas or in liquid form, as in an aqueous solution) produced by hydrogen sulfide generator 91 and is optionally also permeable to liquids such as water and/or a liquid electrolyte solution (which may be an exudate from living tissue 102). Therefore, bottom element 93 is constructed of one or more hydrogen sulfide-permeable materials as described before, and may comprise, for example, a hydrogen sulfide-permeable membrane as described before. In some embodiments, bottom element 93 has pores or other openings that allow bulk movement of hydrogen sulfide and/or other fluids in the form of a gas and/or liquid (such as an aqueous solution of hydrogen sulfide). In such embodiments, bottom element 93 is constructed of one or more liquid-permeable materials, also as described above, such as a gauze or other fabric made up of natural and/or synthetic fibers; a mesh; a porous sheet or film; a polymeric foam, preferably hydrophilic, and the like. Such a gauze, fabric, mesh, porous sheet or film or foam may be impregnated with a hydrogen or a hydrogel precursor that forms a hydrogel upon contact with an aqueous fluid such as a liquid electrolyte solution (such as an exudate from living tissue).

In a particularly preferred embodiment, bottom element 93 is integrated into hydrogen sulfide generator 91 and comprises an electrode/solid-state hydrogen sulfide precursor assembly such as shown, for example, in FIG. 7. In such an embodiment, electrical conductor 72 preferably is a metal that has antimicrobial properties, such zinc, copper, selenium, iron or silver, most preferably silver, and the hydrogen sulfide precursor is the corresponding sulfide, especially silver sulfide. The hydrogen sulfide precursor in such embodiments preferably forms a layer or coating on all or part of electrical conductor 72. The electrode/solid-state hydrogen sulfide precursor assembly preferably is oriented so at least a portion of electrical conductor 72 is in contact with living tissue when applied. In such embodiments, the antimicrobial metal is generated and/or exposed as the hydrogen sulfide precursor is consumed to produce hydrogen sulfide, thereby creating an exposed antimicrobial surface in contact with the underlying living tissue. In an especially preferred embodiment, electrical conductor 72 comprises silver, the hydrogen sulfide generator is silver sulfide in the form of a layer on all or part of electrical conductor 72, and silver metal is produced and/or exposed as hydrogen sulfide is produced.

In the embodiment shown in FIG. 9, optional covering 92 encapsulates exposed surfaces of hydrogen sulfide generator 91. Such an optional covering may form all or part of a housing for the electrolytic cell, or may be a separate or additional covering. Covering 92 can perform various functions, such as being a hydrogen sulfide barrier, a barrier to the loss of moisture, such as electrolyte solution or liquid component thereof; being a mounting surface for various optional components such as electrical contacts 100 and ports 98 as well as various electronic, pumping and/or other useful components, and the like.

The particular tissue dressing shown in FIG. 9 is useful as a vacuum dressing. Gas can be supplied and/or withdrawn through lines 99 which are in fluid communication with ports 98. Pumping apparatus in controller 97 controls the flow of gas through lines 99, ports 98, and into and out of tissue dressing 90 to, for example, produce a vacuum and/or flow other fluids into and/or out of tissue dressing 90. In the embodiment shown in FIG. 9, voltage is applied to hydrogen sulfide generator 91 from controller 97 through wires 101 and electrical contacts 100.

In FIG. 9 controller 97 is shown as a separate module, but if desired it or any component thereof may be integrated into tissue dressing 90. The various functional components of controller 97 may be separated into different modules, each of which may be integrated into tissue dressing 90 as desirable.

Tissue dressing 90 is used by applying it to living tissue 102, with tissue-contact side 95 being brought into contact with living tissue 102. A voltage is then applied across the electrolytic cell of hydrogen sulfide generator 91, applying a negative potential to the first electrode, causing it to function as a cathode and supplying electrons to the hydrogen sulfide precursor, forming hydrogen sulfide. The tissue dressing remains in place so that hydrogen sulfide so produced actively or passively permeates through bottom element 93 of tissue dressing 90 and comes into contact with living tissue 102.

In the particular embodiment shown in FIG. 9, tissue dressing 90 is a vacuum dressing. In such a case, the method optionally further comprises, producing a vacuum between the wound dressing and the tissue. Preferably, the step of producing a vacuum is performed intermittently, and steps of applying a voltage to the hydrogen sulfide generator and permeating hydrogen sulfide to the living tissue are performed between successive iterations of producing a vacuum. Other optional features of the hydrogen sulfide generator and tissue dressing of the invention include, for example, one or more ports for introducing and/or withdrawing electrolyte; mechanical, magnetic and/or adhesive fasters for affixing the hydrogen sulfide generator to another article; and the like. An optional rim such as rim 94 (FIG. 9) can provide a surface for affixing tissue dressing 90 to living tissue 102 and/or forming a seal therebetween. For example, rim 94 may comprise an adhesive layer on its bottom (tissue-side) surface.

Another aspect of the invention is an implantable tissue dressing comprising a hydrogen sulfide generator of the third aspect, such as is shown in FIG. 3. Such an implantable tissue dressing may consist entirely of the hydrogen sulfide generator. Optionally, it may contain one or more optional components.

In FIG. 10, implantable tissue dressing 120 of the invention is implanted within living tissue 102. Lead wires 121 provide an electrical connection with a remote apparatus for applying a voltage across the first and counter-electrodes. Alternatively, such apparatus may be contained within implantable tissue dressing 120, or implanted therewith. For example, an all-hydrogel battery as described in Adv. Mater. 2021, 205120, may be incorporated within implantable tissue dressing 120 or implanted as a separate component, to serve as all or part of the voltage supplying apparatus.

At least the surface of such an implantable tissue dressing preferably is biocompatible. Thus, referring again to FIG. 3, if gel electrolyte 208 forms an exposed surface of the implantable tissue dressing, gel electrolyte 208 is biocompatible. Some or all of the exterior surface of gel electrolyte 208 may be completely or partially covered with a hydrogen sulfide-permeable surface layer (not shown) that forms all or part of an exposed exterior surface of hydrogen sulfide generator 201; in such a case, such surface layer is biocompatible. In especially preferred embodiments, the gel electrolyte 208 and any hydrogen sulfide-permeable surface layer are both biocompatible and bioresorbable.

In especially preferred embodiments of an implantable tissue dressing, the first and counter electrodes are (1) intrinsically conductive biocompatible and preferably bioresorbable polymers, (2) comprise particles of a biocompatible metal, the particles optionally being composite particles further containing a hydrogen sulfide precursor, dispersed in a biocompatible and preferably bioresorbable binder, and/or (3) comprise particles of a biocompatible metal and particles of a hydrogen sulfide precursor dispersed in a biocompatible and preferably bioresorbable binder. The gel electrolyte and any surface layer thereupon in such especially preferred embodiments also preferably are biocompatible and more preferably both biocompatible and bioresorbable.

In another especially preferred embodiment of the implantable tissue dressing, the hydrogen sulfide precursor is at least partially present in the form of particles dispersed in a biocompatible and preferably bioresorbable binder, preferably a gel binder, the hydrogen sulfide precursor and binder being separate from but in electrical contact with at the first electrode and optionally the counter-electrode. The gel electrolyte and any surface layer thereupon in such other especially preferred embodiments also preferably are biocompatible and more preferably both biocompatible and bioresorbable, and the first and counter electrodes are (1) intrinsically conductive biocompatible and preferably bioresorbable polymers, (2) comprise particles of a biocompatible metal, the particles optionally being composite particles further containing a hydrogen sulfide precursor, dispersed in a biocompatible and preferably bioresorbable binder, and/or (3) comprise particles of a biocompatible metal and particles of a hydrogen sulfide precursor dispersed in a biocompatible and preferably bioresorbable binder.

An implantable tissue dressing of the invention is implanted into living tissue, surgically or otherwise. The implantable tissue dressing may be sutured in place, mounted on a scaffold (preferably a bioresorbable scaffold), or otherwise secured in place if necessary. A voltage is applied across the electrolytic cell of the implantable tissue dressing to convert at least a portion of the sulfur-containing hydrogen sulfide precursor to hydrogen sulfide. The hydrogen sulfide permeates out of the implantable tissue dressing and into the surrounding living tissue.

In especially preferred embodiments in which the gel electrolyte, any surface covering thereon, any polymeric electrode and any binder present (including any binders present in the electrodes and those used to separately bind particles of the hydrogen sulfide precursor) are all bioresorbable, the implantable tissue dressing can be made to be almost entirely bioresorbable. In such embodiments, essentially the entire implantable tissue dressing can become resorbed by the living tissue. Metal particles from the electrode(s) may or may not be resorbable, but in such embodiments such particles preferably are biocompatible and more preferably are biocompatible metals such as zinc, copper, selenium, iron or silver, most preferably silver. Small residues of these particles as may remain after of the implantable tissue dressing are often tolerated by living organisms, particularly mammals such as humans. Such residual particles even can play a beneficial role, such as an antimicrobial function. Thus, in certain preferred embodiments, the implantable tissue dressing of the invention is not removed from the tissue after use but is allowed to remain therein and be bioresorbed.

FIG. 11 illustrates an embodiment of another aspect of the invention. Hydrogen sulfide generator 300, implanted in living tissue 102, includes first wire electrode 301 having at least one solid-state sulfur-containing hydrogen sulfide precursor incorporated into and/or forming a layer on a surface of the first wire electrode. At least a portion of first wire electrode 301 or solid-state sulfur-containing hydrogen sulfide precursor is exposed at a surface of the hydrogen sulfide generator. Wire counter-electrode 303 and first wire electrode 301 are parallel in the embodiment shown in FIG. 1, but in other embodiments they may be braided or twisted together. Gel electrolyte 304 separates first wire electrode 301 and wire counter-electrode 303. In the embodiment shown, gel electrolyte 304 takes to the form of discontinuous segments coated onto the surface of counter-electrode 303. Alternatively, gel electrolyte 304 may form a continuous layer on counter-electrode 303, or may be a separate component interposed between and separating first wire electrode 301 and wire counter-electrode 303. The compositions of wire electrode 301, wire counter-electrode 303 and gel electrolyte 304 are generally as described before. As before, hydrogen sulfide generator of this aspect of the invention is operated by applying a voltage across first wire electrode 301 and wire counter-electrode 303. Accordingly, hydrogen sulfide generator 300 includes apparatus for applying such a voltage or connection means for connecting hydrogen sulfide generator 300 to such apparatus. A hydrogen sulfide generator such as shown in FIG. 11 is useful as a suture such as a surgical suture) which upon application of a voltage delivers hydrogen sulfide to adjacent tissue.

Turing to FIG. 12, hydrogen sulfide generator 40, suitable as an implantable tissue dressing, comprises first electrode 401 and counter-electrode 402. As before, solid-state hydrogen sulfide precursor is in electrical contact with at least first electrode 401 and preferably both first electrode 401 and counter-electrode 402. First electrode 401 and counter-electrode 402 are spaced apart and separated by electrolyte 403, which is preferably a gel electrolyte and/or a liquid electrolyte contained in a porous spacer medium such as an open-celled polymer foam. Tube 404 encapsulates first electrode 401, counter-electrode 402 and electrolyte 403. Tube 404 is preferably permeable to hydrogen sulfide but impermeable to electrolyte 403; a silicone tube is suitable. Tube 404 may be a biocompatible and/or bioresorbable material, provided it is permeable to hydrogen sulfide. Electrolyte 403 and/or any porous spacer medium also optionally are biocompatible and/or bioresorbable. Electrodes 401 and 402 each may be composite electrodes such as shown in FIGS. 4, 5, 8 and/or 8A. A separator as described before may be interposed between first electrode 401 and counter-electrode 402.

An important advantage of this invention, when used as or as a component of a tissue dressing, it that very small, therapeutic levels of hydrogen sulfide can be generated over an extended period of time of up to days or weeks, using very small applied voltages and currents. A tissue dressing of the invention may contain, for example as little as 0.1 micromoles of hydrogen sulfide precursor, and still generate therapeutic levels of hydrogen sulfide for application to a wound. A preferred amount of hydrogen sulfide is, for example, at least 0.1 micromole, at least 0.5 micromoles or at least 1 micromole, and up to as much as 1 millimole, 0.1 millimole or 0.05 millimole. A hydrogen sulfide generator of the invention used in a tissue dressing may generate, for example, 5 to 1000 nanomoles of hydrogen sulfide per day of continuous operation or, on a per-minute basis, about 0.004 to a about 0.7 nanomoles per minute of operation. In preferred cases in which the hydrogen sulfide precursor is silver sulfide, the silver produced as hydrogen sulfide is generated provides an added antimicrobial benefit to a tissue dressing.

### EXAMPLES

The following examples are provided to illustrate the invention but are not intended limit the scope thereof. All parts and percentages are by weight unless otherwise indicated.

### Example 1

Liver of sulfur is diluted with water in a 1:30 volume ratio. A length of square silver wire is immersed in the liver of sulfur solution at room temperature in a separated conical tube. The surface of the silver wire turns black as it becomes sulfurized to produce a silver sulfide layer on the wire surface.

A hydrogen sulfide generator is produced by assembling the sulfurized silver wire as the first electrode and another length of the sulfurized silver wire as the counter-electrode within a polypropylene box. A gelatin methacryloyl (GelMa)/saline solution hydrogen (0.9% sodium chloride in the saline solution) is the electrolyte. A hydrogen sulfide-permeable silicone membrane is stretched across the open end of the polypropylene box to seal the contents and produce a liquid barrier. The cell includes a headspace for collection of hydrogen sulfide. Hydrogen sulfide produced by the cell diffuses through the silicone membrane and is captured in the headspace.

The hydrogen sulfide generator is evaluated by applying a voltage of about −1V to the first electrode. Gas samples from the headspace are collected at intervals of five minutes and transferred to an electrochemical hydrogen sulfide analyzer. The cell produces about 3.5 to 6.3×10⁻⁹ moles of hydrogen sulfide per minute. The H₂S production rate varies approximately linearly with small variations in the applied voltage (and hence total current per unit time). This rate of hydrogen sulfide is appropriate for administration at a wound side to promote vascularization.

### Example 2

A hydrogen sulfide generator is constructed within a polypropylene conical tube having ports for two electrode connections and 2 ports for introducing and removing gas from a headspace. The first electrode is a sulfurized silver wire as described in Example 1. The counter electrode is an unsulfurized length of the silver wire. The electrolyte is an over-the-counter eye irrigation saline solution containing about 0.9% sodium chloride. No silicone barrier is present. A polypropylene mesh separator is interposed between the electrodes. A headspace is provided for collection of hydrogen sulfide.

The hydrogen sulfide generator is evaluated in the general manner described in Example 1 and confirms that hydrogen sulfide is produced by the cell.

### Example 3

A woven conductive fabric plated with copper/nickel, having a thickness of 0.08 mm (Adafruit P1168, Adafruit, New York, NY), is sulfurized in a diluted liver of sulfur solution in the general manner described in Example 1. This produces a copper sulfide coating on the surface of the metal.

A hydrogen sulfide generator is constructed within a polypropylene conical tube as described in Example 2. The first electrode is the sulfurized conductive fabric. The counter electrode is an unsulfurized length of the silver wire described in Example 1. The electrolyte is a 0.05M sulfuric acid solution. A polypropylene mesh separator is interposed between the electrodes. The hydrogen sulfide generator is evaluated in the general manner described in Example 1. Hydrogen sulfide generation is detected upon application of a voltage of −0.5 to −2 V to the first electrode.

### Example 4

A commercial antimicrobial silver dressing (Silverlon® from Argentum Medical, LLC) containing 546 mg of silver per 100 cm² sample is sulfurized in the general manner described in Example 1 to introduce a silver sulfide coating onto the silver contained within the dressing. The dressing turns black in color as silver sulfide is produced. The sulfurization time is restricted to a few minutes as complete conversion of the silver in the dressing to silver sulfide destroys the electrical conductivity of the dressing.

A hydrogen sulfide generator is constructed in a polypropylene housing using a section of the sulfurized dressing as the first electrode, a length of the unsulfurized silver wire described in Example 1 as the counter-electrode, a polypropylene mesh separate interposed between the electrodes and sulfuric acid solutions ranging from 0.001M to 2M as the electrolyte. The hydrogen sulfide generator produces about 298 nanomoles of hydrogen sulfide per hour at an applied voltage of −1V (to the first electrode).

### Example 5

A 30 ga. 99.999% fine round silver wire is sulfurized in the general manner described in Example 1.

A hydrogen sulfide generator is made by wrapping a porous polypropylene mesh separator around a gold wire (which serves as the counter-electrode). The separator is wetted with an over-the-counter eye saline solution containing sodium chloride, boric acid, sodium borate, potassium chloride, sodium chloride and biocide. The sulfurized silver wire is wrapped around the separator. This assembly is inserted into a hydrogen sulfide-permeable silicone tube.

The hydrogen sulfide generator is placed in a sealed container. The electrodes are attached to a potentiostat. A voltage of −2V is applied to the sulfurized silver electrode using the gold electrode as the counter-electrode. Periodic analysis of gas accumulating in the sealed container confirms that hydrogen sulfide is generated by and released from the hydrogen sulfide generator.

1-2. (canceled)

3. A hydrogen sulfide generator comprising an electrolytic cell, the electrolytic cell comprising A) a first electrode, B) at least one solid-state sulfur-containing hydrogen sulfide precursor in electrical contact with the first electrode, (C) a counter-electrode spaced apart from (i) the first electrode and (ii) the at least one solid-state sulfur-containing hydrogen sulfide precursor in electrical contact with the first electrode, and D) an electrolyte comprising saline and/or a mammalian bodily fluid disposed between the first and counter-electrode, wherein the hydrogen sulfide generator is permeable to hydrogen sulfide produced by the electrolytic cell such that hydrogen sulfide produced by the electrolytic cell is permeable out of at least one surface of the hydrogen sulfide generator, wherein the at least one solid-state sulfur-containing hydrogen sulfide precursor is insoluble in the electrolyte and chemically stable in the presence of the electrolyte in the absence of an applied voltage to the electrolytic cell.

4-10. (canceled)

11. The hydrogen sulfide generator of claim 3, wherein the electrolyte is devoid of a sulfur-containing hydrogen sulfide precursor.

12. The hydrogen sulfide generator of claim 3, wherein the solid-state sulfur-containing hydrogen sulfide precursor forms a layer on at least a portion of a surface of the first electrode.

13-14. (canceled)

15. The hydrogen sulfide generator of claim 3 further comprising apparatus for applying a voltage across the electrolytic cell.

16. (canceled)

17. The hydrogen sulfide generator of claim 3 wherein the at least one sulfur-containing hydrogen sulfide precursor is a compound comprising a metal and sulfur.

18-19. (canceled)

20. The hydrogen sulfide generator of claim 17 wherein the first electrode comprises a metal M, and the solid-state sulfur-containing hydrogen sulfide precursor comprises a sulfide of the metal M, and the metal M is one or more of silver, copper, zinc, selenium and iron.

21. (canceled)

22. The hydrogen sulfide generator of claim 3 wherein the first electrode comprises electrically conductive particulates embedded in a binder that is biocompatible, bioresorbable, or both biocompatible and bioresorbable.

23. (canceled)

24. The hydrogen sulfide generator of claim 22, wherein particles of the at least one solid-state sulfur-containing hydrogen sulfide precursor are embedded in the binder of the first electrode.

25. The hydrogen sulfide generator of claim 22 wherein the electrically conductive particles of the first electrode are coated with the solid-state sulfur-containing hydrogen sulfide precursor.

26-60. (canceled)

61. A hydrogen sulfide generator comprising an electrolytic cell, the electrolytic cell comprising A) a first electrode comprising electrically conductive particles embedded in a binder that is biocompatible, bioresorbable, or both biocompatible and bioresorbable, B) at least one solid-state sulfur-containing hydrogen sulfide precursor in electrical contact with the first electrode, (C) a counter-electrode spaced apart from (i) the first electrode and (ii) the at least one solid-state sulfur-containing hydrogen sulfide precursor in electrical contact with the first electrode, and D) an electrolyte disposed between the first and counter-electrode, wherein the hydrogen sulfide generator is permeable to hydrogen sulfide produced by the electrolytic cell such that hydrogen sulfide produced by the electrolytic cell is permeable out of at least one surface of the hydrogen sulfide generator, wherein the at least one solid-state sulfur-containing hydrogen sulfide precursor is insoluble in the electrolyte and chemically stable in the presence of the electrolyte in the absence of an applied voltage to the electrolytic cell.

62. The hydrogen sulfide generator of claim 61, wherein particles of the at least one solid-state sulfur-containing hydrogen sulfide precursor are embedded in the binder of the first electrode.

63. The hydrogen sulfide generator of claim 62, wherein the electrically conductive particles of the first electrode are coated with the solid-state sulfur-containing hydrogen sulfide precursor.

64. The hydrogen sulfide generator of claim 61 wherein the counter-electrode comprises electrically conductive particles embedded in a binder which is biocompatible, bioresorbable, or both biocompatible and bioresorbable.

65. The hydrogen sulfide generator of claim 61 further comprising apparatus for applying a voltage across the electrolytic cell.

66. A tissue dressing comprising a hydrogen sulfide generator comprising an electrolytic cell, the electrolytic cell comprising A) a first electrode, B) at least one solid-state sulfur-containing hydrogen sulfide precursor in electrical contact with the first electrode, (C) a counter-electrode spaced apart from (i) the first electrode and (ii) the at least one solid-state sulfur-containing hydrogen sulfide precursor in electrical contact with the first electrode, and D) an electrolyte disposed between the first and counter-electrode, wherein the hydrogen sulfide generator is permeable to hydrogen sulfide produced by the electrolytic cell such that hydrogen sulfide produced by the electrolytic cell is permeable out of at least one surface of the hydrogen sulfide generator, wherein the at least one solid-state sulfur-containing hydrogen sulfide precursor is insoluble in the electrolyte and chemically stable in the presence of the electrolyte in the absence of an applied voltage to the electrolytic cell.

67. The tissue dressing of claim 66 which is a wound or surgical dressing, a catheter dressing, an implantable dressing or a surgical suture.

68. The tissue dressing of claim 66 further comprising a support, the tissue dressing having a tissue-contact side and an opposing side, wherein at least a portion of the surface of the tissue-contact side of the tissue dressing is permeable to hydrogen sulfide produced by the electrocatalytic cell of the hydrogen sulfide generator.

69. The tissue dressing of claim 68 wherein the support comprises one or more of a gauze or other fabric made up of natural and/or synthetic fibers, a mesh, a porous sheet or film and a polymeric foam.

70. The tissue dressing of claim 61 wherein the electrolyte comprises saline, a mammalian bodily fluid and/or a gel electrolyte.

71. A tissue treatment method comprising
a) applying a tissue dressing of claim 61 to living tissue;
b) applying a voltage across the electrolytic cell of the tissue dressing to convert at least a portion of the sulfur-containing hydrogen sulfide precursor to hydrogen sulfide; and
c) actively or passively permeating the hydrogen sulfide through the tissue dressing and into contact with the living tissue.
